(19) United States
(12) Patent Application Publication
(10) Pub. No.: US 2021/0128724 A1
(43) Pub. Date: May 6, 2021

(54) CGRP ANTAGONISTS AND CLOSTRIDIAL DERIVATIVES FOR THE TREATMENT OF CORTICAL SPREADING DEPRESSION ASSOCIATED DISORDERS

(71) Applicant: Allergan Pharmaceuticals International Limited, Dublin 17 (IE)

(72) Inventors: Rami Burstein, Chestnut Hill, MA (US); Agustin Melo Carrillo, Boston, MA (US); Mitchell F. Brin, Newport Beach, CA (US); Andrew M. Blumenfeld, Del Mar, CA (US)

(21) Appl. No.: 16/921,530

(22) Filed: Jul. 6, 2020

Related U.S. Application Data
(60) Provisional application No. 62/870,911, filed on Jul. 5, 2019.

(51) Int. Cl. A61K 39/395; A61K 38/48; A61K 31/4545; A61K 31/485; A61K 31/4468; A61K 31/451; A61K 31/439; A61K 31/4535; A61K 31/137; A61K 31/135; A61K 31/55; A61K 31/4453; A61K 31/454; A61K 31/167; A61K 31/192; A61K 31/616; A61K 9/00; A61P 25/28; A61P 25/06; A61P 25/00; C07K 16/18; C07K 16/22

(52) U.S. Cl. CPC A61K 39/3955; A61K 2039/545; A61K 31/4545; A61K 31/485; A61K 31/4468; A61K 31/451; A61K 31/439; A61K 31/4535; A61K 31/137; A61K 31/135; A61K 31/55; A61K 31/4453; A61K 31/454; A61K 31/167; A61K 31/192; A61K 31/616; A61K 9/0019; A61P 25/28; A61P 25/06; A61P 25/00; C07K 16/18; C07K 16/22; A61K 38/4893

(57) ABSTRACT

Methods for the treatment and prevention of diseases and disorders associated with cortical spreading depression by administering a CGRP antagonist, a Clostridial derivative or combination thereof are described.

### CROSS-REFERENCES TO RELATED APPLICATION

This application claims priority pursuant to 35 U.S.C. § 119(e) to U.S. Provisional Patent Application Ser. No. 62/870,911 filed Jul. 5, 2019, which is incorporated entirely herein by reference.

### FIELD

The present disclosure relates to medicaments and methods for the treatment of diseases and disorders associated with cortical spreading depression. In particular, the present application is directed to methods for treating diseases and disorders associated with cortical spreading depression by administration of a clostridial derivative, a calcitonin-gene-related-peptide (hereafter referred to as CGRP) antagonist, or combination thereof.

### BACKGROUND

Cortical spreading depression (CSD) has been shown to occur as a wave of depolarization in the cerebral cortex that propagates slowly across the brain at approximately 1.7 to 9.2 millimeters/minute. The depolarization wave is characterized by dramatic increases in K⁺ and glutamate extracellularly and increases in Na⁺ and Ca²⁺ intracellularly. The depolarization leads to a sustained suppression or depression of nerve cell activity from which the phenomenon takes its name. In individuals without overt brain damage, normal brain activity then resumes. However, spreading depolarization waves also occur in patients with developing brain damage (e.g., post stroke) whose brain activity is already depressed; in such cases, the phenomenon has been called cortical spreading depolarization to indicate that the depression wave may not be evident on electroencephalography (EEG) due to initial depression of activity.

CSD and/or spreading depolarization is observed in a variety of serious medical conditions, including acute brain injury and/or ischemia (e.g., from acute physical trauma, stroke, subarachnoid or intracranial hemorrhage, cardiac arrest, etc.), as well as migraine, migraine variants (e.g., status migrainosus, persistent aura without infarction, migrainous infarction, etc.), and seizures. Spreading depolarization is essential to the development of brain lesions, and in conditions of severe ischemia and hemorrhage, CSD and spreading depolarizations expand brain lesions causing ongoing, secondary damage that can last for hours. In migraine, CSD is the presumed neuropathological correlate of aura—sensory changes that precede migraine headaches, leading to inflammation that can eventually trigger headaches. Spreading depolarization may limit brain recovery following seizures, and seizures can trigger spreading depolarization, possibly leading to death.

Thus, there is a need for treatment methods that reduce the occurrences and/or propagation of CSD and/or spreading depolarization and thereby treat, prevent and/or alleviate cortical spreading depression associated disorders and/or symptoms thereof.

### SUMMARY

Methods for treating, preventing, alleviating or reducing the intensity or frequency of occurrence of a condition associated with cortical spreading depression (CSD) and/or symptoms thereof are provided.

In one aspect, a method for treating, preventing, alleviating or reducing the intensity or frequency of a CSD-related disorder and/or symptoms thereof is provided, the method comprises administering to a subject with a CSD associated disorder a calcitonin gene-related peptide antagonist (CGRP-antagonists), a clostridial derivative, or combination thereof. In some embodiments, the administration of the combination produces a synergistic effect relative to treatment with the CGRP antagonist or the clostridial derivative alone.

In some embodiments, the CGRP antagonist is a targeted CGRP small molecule antagonist. In some embodiments, the CGRP-antagonist is ubrogepant, atogepant, or a pharmaceutically acceptable salt, ester or prodrug thereof. In alternative embodiments, the CGRP-antagonist is an antibody. In some embodiments, the CGRP-antagonist antibody is selected from galcanezumab, fremanezumab, eptinezumab, and erenumab.

In some embodiments, the clostridial derivative is a botulinum toxin. In other embodiments, the clostridial derivative is a Targeted Exocytosis Modulators (TEMS).

In some embodiments, the CSD-associated disorder comprises migraine and migraine variants, stroke and other causes of brain ischemia, subarachnoid and intracranial hemorrhage, traumatic brain injury, seizures, head injury/head trauma (concussion), post traumatic stress disorder, fibromyalgia and tinnitus (either associated with or not associated with another disorder). In some embodiments, the CSD associated disorder is selected from stroke and other causes of brain ischemia, subarachnoid and intracranial hemorrhage, traumatic brain injury, seizures, and head injury/head trauma (concussion). In some embodiments, the CSD-associated symptoms are other than headache or migraine.

In some embodiments, the method comprises preventing, alleviating or reducing the intensity or frequency of occurrence of a CSD-related disorder and/or symptom in a patient in need thereof. In some embodiments, the CSD-related disorder is selected from stroke and other causes of brain ischemia, subarachnoid and intracranial hemorrhage, traumatic brain injury, seizures, head injury/head trauma (concussion), post-traumatic stress disorder after a head trauma and fibromyalgia another aspect, there is provided a method for preventing, alleviating or reducing the intensity or frequency of occurrence of a symptom related to or resulting from a CSD associated disorder, the method comprises administering to a patient with the CSD associated disorder a calcitonin gene-related peptide antagonist (CGRP-antagonists), a clostridial derivative, or combination thereof. In some embodiments, the administering is carried out between an initial time when the CSD associated disorder occurs and the onset of the resulting CSD associated symptom. In some embodiments, the administering is carried out after the occurrence of the CSD associated disorder and before the onset of the resulting CSD associated symptom. In some embodiments, the administration of the combination produces a synergistic effect relative to treatment or prevention with the CGRP antagonist or the clostridial derivative alone. In some embodiments, the CSD associated disorder is selected from stroke and other causes of brain ischemia, subarachnoid and intracranial hemorrhage, traumatic brain injury, seizures, head injury/head trauma (concussion), post-traumatic stress disorder after a head trauma and fibromyalgia.

In some embodiment, the CSD associated disorder is a traumatic brain injury. In some embodiments, the method comprises administering a CGRP-antagonist, a clostridial derivative or combination thereof between the time when the traumatic brain injury occurs and the onset of the TBI associated/related symptom. In some embodiments, the administering is carried out after the occurrence of the traumatic brain injury and before the onset of the TBI related symptom. In some embodiments, the administering is carried out within four months after the traumatic brain injury. In some embodiments, the administering is carried out within 1 hour, 2 hours, 3 hours, 6 hours, 12 hours, 24 hours, 48 hours or 72 hours of the traumatic brain injury. In alternative embodiments, the administering is carried out within 1 week, two weeks, three weeks, four weeks, five weeks, six weeks, seven weeks, eight weeks, nine weeks, ten weeks, eleven weeks, twelve weeks, thirteen weeks, fourteen weeks, fifteen weeks, sixteen weeks, or up to 24 weeks of the traumatic brain injury. In some embodiments, the administering is carried out prior to onset of at least one of the traumatic brain injury associated symptoms. In some embodiments, the administering is carried out within two months after the onset of a first traumatic brain injury associated symptom. In some embodiments, the traumatic brain injury associated symptoms comprise hypotension, hypoxemia, brain swelling, headache, mental fog, decreased attention, decreased reaction time, neck pain, a difficulty remembering, a difficulty concentrating, a difficulty making decisions, fatigue, a mood change, nausea, sleep disturbance, mild motor disturbance, photophobia, blurred vision, ear ringing (tinnitus), a loss of sense of taste, and a loss of sense of smell, seizures, coma, muscle weakness, paralysis, memory loss, sexual dysfunction, cognitive impairment and a progressive decline in neurologic function. In some embodiments, the administering is carried out in the period following the traumatic brain injury (TBI) and before the development of post traumatic stress disorder (PTSD), which typically occurs about 3 months after the injury. In some embodiments, the administering is carried out in the period following the traumatic brain injury (TBI) and before the development of headache, including migraine and migraine variants.

In some embodiments, the administration of the combination produces a synergistic effect relative to treatment or prevention with the CGRP antagonist or the clostridial derivative alone.

### BRIEF DESCRIPTION OF THE DRAWINGS

The following drawings are presented to illustrate aspects and features of embodiments of the present screening assay and methods.

FIG. 1 shows responses of C-fibers to CSD followed treatment with an exemplary clostridial derivative, BoNT/A (onabotulinumtoxinA), in accordance with aspects of the present disclosure. Plots of firing rate before and after induction of CSD, for two C-fibers with CSD responses in a saline-treated (upper) and a BoNT-A-treated (middle) animal, and for a C-fiber that showed no CSD response (lower). The horizontal dotted line marks the firing level two standard deviations above the baseline rate;

FIG. 2 shows responses of A delta-fibers to CSD followed treatment with the exemplary clostridial derivative, onabotulinumtoxinA, in accordance with aspects of the present disclosure. Plots of firing rate before and after induction of CSD, for two A-delta-fibers with CSD responses in a saline-treated (upper) and a BoNT-A-treated (middle) animal, and for an A-delta-fiber that showed no CSD response (lower). The horizontal dotted line marks the firing level two standard deviations above the baseline rate;

FIG. 3 shows CSD response amplitude. Box-and-whisker plots of amplitude of CSD responses for C-fibers (A) and A delta-fibers (B) in saline- and BoNT-A-treated animals. Response amplitude was quantified as the activity that was more than 2 standard deviations above the baseline rate, during the interval 60-180 minutes post-CSD, and is expressed here as total spikes;

FIG. 4 shows mean neuronal firing rate of high threshold (HT) neurons before and after cortical spreading depression (CSD) in control animals vs. those treated with a combination of an exemplary clostridial derivative, BoNT-A (onaboutlinumtoxinA) and an exemplary CGRP-antagonist, Atogepant in accordance with aspects of the present disclosure; and

FIG. 5 shows mean neuronal firing rate of wide dynamic range (WDR) neurons neurons before and after cortical spreading depression (CSD) in control animals vs. those treated with the combination of an exemplary clostridial derivative, onaboutlinumtoxinA and an exemplary CGRP-antagonist, Atogepant in accordance with aspects of the present disclosure.

### DESCRIPTION

The application provides methods for treating, preventing, alleviating or reducing the intensity or frequency of occurrence of a disease or disorder associated with CSD and/or symptoms thereof in patients by the use antagonists of calcitonin gene-related peptide (CGRP-antagonist), a clostridial derivative, or combination thereof. CSD-related disorders include but are not limited to migraine or migraine variants, stroke and other causes of brain ischemia, subarachnoid and intracranial hemorrhage, traumatic brain injury, seizures, and head injury/head trauma (concussion). The present disclosure provides methods for the treatment of one or more CSD-related diseases or conditions. In some embodiments, the CSD-related disorder or condition comprises migraine or migraine variants, stroke and other causes of brain ischemia, subarachnoid and intracranial hemorrhage, traumatic brain injury, seizures, and head injury/head trauma (concussion) and/or symptoms thereof. In some embodiments, the CSD-related disorder or condition is selected from stroke and other causes of brain ischemia, subarachnoid and intracranial hemorrhage, traumatic brain injury, seizures, head injury/head trauma (concussion), post-traumatic stress disorder (PTSD), fibromyalgia, and/or symptoms thereof. In some embodiments, the CSD-related symptoms are other than headache, migraine or migraine variants.

CSD-Related Disorders

Seizure:

Seizures are generally associated with abnormal paroxysmal electrical brain activity. The clinical manifestation of seizures may include alterations of consciousness and motor, sensory, autonomic, or psychic events perceived by the patient or an observer. Often, seizures result in temporary loss of consciousness, convulsions, changes in muscle tone, unusual movements and/or staring spells. Seizures can have many causes, such as medicines, high fevers, head injuries, genetic etiologies, infectious diseases, metabolic disorders, stroke, and other diseases. Recurrent unprovoked seizures define the neurologic syndrome known as epilepsy. Seizure can be diagnosed by observation, or by recording of brain electrical activity, as by an electroencephalogram (EEG).

Seizures are divided into three categories based on the symptoms that a patient or an observer perceive, and/or the pattern of electrical brain activity that accompanies a seizure. In broadest terms, seizures can be classified as focal, generalized and unclassified.

Focal (also called “partial”) seizures are those in which the initial semiology or EEG findings indicate onset in only part of one cerebral hemisphere. Notably, focal seizure often arises with asymptomatic abnormal electrical brain activity in only a small volume of cortex, in one hemisphere. Pursuant to their very localized onset, focal seizures spread to adjacent cortical regions within the hemisphere, at which time they are perceived by the patient or observer. Symptoms of partial seizures include, but are not limited to, abnormal muscle contraction, staring spells, forced eye movements, abnormal sensations such as numbness and tingling, hallucinations, abdominal pain, nausea, abnormal sweating, rapid heart rate, vision changes, mood changes, and loss of consciousness. Partial seizures are generally classified as “simple” or “complex,” based on observed symptoms, analysis of electroencephalographic (EEG) data, or other factors.

Generalized seizures are those with an initial semiology or EEG findings indicate more than minimal involvement of both cerebral hemispheres and may be subdivided into convulsive seizures and non-convulsive seizures.

Another subcategorization of generalized seizures is into those are primary generalized and arise simultaneously from both hemispheres, and those that are secondarily generalized and arise focally in one hemisphere before spreading to involve the other hemisphere. The third category, unclassified seizures, includes all seizures that defy classification due to incomplete data.

Non-limiting examples of seizures include epilepsy, juvenile myoclonic epilepsy, idiopathic generalized epilepsy, and Lennox-Gastaut syndrome. In another embodiment, the seizures experienced by the subject to be treated are associated with an epilepsy syndrome, including epilepsy, or other disorder characterized by seizures. Non-limiting examples of epilepsy syndromes and other disorders associated with seizures include benign familial neonatal seizures, early myoclonic encephalopathy, Ohtahara syndrome, migrating partial seizures of infancy, West syndrome, benign myoclonic epilepsy in infancy, benign familial infantile seizures, benign infantile seizures (nonfamilial), Dravet syndrome, hemiconvulsion-hemiplegia syndrome, benign childhood epilepsy with centrotemporal spikes, early-onset benign childhood occipital epilepsy (Panayiotopoulos type), late-onset childhood occipital epilepsy (Gastaut type), epilepsy with myoclonic absences, epilepsy with myoclonic-astatic seizures, Lennox-Gastaut syndrome, Landau-Kleffner syndrome (LKS), epilepsy with continuous spike-and-waves during slow-wave sleep (other than LKS), childhood absence epilepsy, progressive myoclonus epilepsies, idiopathic generalized epilepsies with variable phenotypes, juvenile absence epilepsy, juvenile myoclonic epilepsy, epilepsy with generalized tonic-clonic seizures only, reflex epilepsies, idiopathic photosensitive occipital lobe epilepsy, startle epilepsy, autosomal dominant nocturnal frontal lobe epilepsy, familial temporal lobe epilepsies, generalized epilepsies with febrile seizures plus, familial focal epilepsy with variable foci, symptomatic focal epilepsies, limbic epilepsies, mesial temporal lobe epilepsy with hippocampal sclerosis, Mesial temporal lobe epilepsy defined by specific etiologies.

Epilepsy affects 1% of the world population. In individuals with epilepsy experiencing localized (focal or partial) seizures, awareness may be disturbed and variable degrees of amnesia may be evident, but the seizure does not lead to loss of consciousness. Focal seizures can, however, progress to secondarily generalized seizures, in which case the individual loses consciousness. Clinically, generalized and bilateral seizures are considered more dangerous because of their greater potential for injury and post-seizure complications. Epidemiologically, nearly half of all adults diagnosed with epilepsy experience at least one generalized or bilateral seizure, and about 25% of patients with epilepsy experience them regularly. Current treatments for epilepsy and other seizures include antiepileptic drugs (AED). However, standard-of-care pharmacotherapy is incompletely effective for many patients and can result in unwanted side effects.

Aura, aura seizure symptoms, or epileptic aura symptoms refer to any warning sensation perceived by a patient that is indicative of an oncoming seizure or epilepsy, which may include, for example, nausea, visual changes, auditory changes, olfactory changes, perception of a strange light, tearfulness of the eyes, an unpleasant smell (phantosmia) or tastes (gustatory hallucinations), synesthesia, cephalic aura, confusing thoughts or experiences, sudden feeling of anxiety, fear or vertigo, numbness or tingling sensation, and hallucinations, etc. The methods disclosed here include treatment of one or more seizure associated symptoms or aura seizure symptoms.

Migralepsy includes the combination of migraine aura acting as a trigger for a seizure. These patients typically have visual aura followed by seizure. The methods disclosed here include treatment of one or more migralepsy associated symptoms.

Stroke:

A stroke may cause a wide variety of symptoms and/or complications. Without wishing to be held to a particular theory, it is thought that the specific signs, symptoms and/or complications, as well as their severity and duration, may be determined by where the stroke occurs in a brain as well as how severe it is. Because the brain controls or modulates nearly every system in the body, the range of potential signs, symptoms and/or complications is vast. In some embodiments, the symptoms arising from a stroke include one or more of neurological impairment, cognitive impairment, language impairment, emotional impairment (e.g., depression, anxiety), and motor impairment. The symptoms include paralysis, memory loss, pain, seizure, dysphagia (difficulty swallowing), aphasia (loss of speech or language ability), dysarthria (difficulty articulating words), ataxia (lack of coordinated movements), and loss of vision. In some embodiments, paralysis is partial (e.g., limited to one muscle group, area of the body, and/or side of the body). In some embodiments, paralysis is substantially complete (e.g., affecting both sides of the body and most or all voluntary muscles below the neck). The methods disclosed here include treatment of one or more stroke associated symptoms.

Subarachnoid and Intracranial Hemorrhage:

Intracerebral hemorrhage (ICH) accounts for approximately 10-15% of all stroke cases. As blood spreads from the point of origin of an ICH through the brain, it can cause infections, high fever, headaches, vomiting, increased blood pressure, hyperglycemia (even in patients without diabetes), seizures, decreased consciousness, blood clots, and events related to blood clots. Neurological symptoms of putaminal bleeding include rapidly appearing hemiplegia, sensory disturbance, homonymous hemianopsia, contralateral conjugate deviation, higher order cerebral function disease and headache. Subarachnoid hemorrhage is due to the rupture of cerebral aneurysm and refers to the state that hemorrhage has occurred in the subarachnoid cavity. Complications of subarachnoid hemorrhage include rebleeding, cerebral vasospasm and hydrocephalus. The methods disclosed here include treatment of one or more symptoms or complications of hemorrhage.

Traumatic Brain Injury and Concussions:

Symptoms of the traumatic brain injury (TBI) include hypotension, hypoxemia, brain swelling, headache, a neck pain, a difficulty remembering, a difficulty concentrating, a difficulty making decisions, fatigue, a mood change, nausea, photophobia, blurred vision, ear ringings, a loss of sense of taste, and a loss of sense of smell, seizures, coma, muscle weakness, paralysis, memory loss, cognitive impairment and a progressive decline in neurologic function following the traumatic brain injury. The concussion can result in loss of consciousness and the post-concussive symptoms can include headache, mental fog, decreased attention, decreased reaction time, concentration, sleep disturbance, mild motor disturbance, delayed complication of post traumatic stress disorder (PTSD), or any combination thereof. The methods described herein can be used to treat, prevent, reduce the intensity or occurrence of one or more symptoms or complications related to traumatic brain injury and concussion.

In one aspect, the present disclosure provides a method for treating, preventing, reducing the intensity or occurrence of a CSD-related disorder and/or symptoms thereof, the method comprises administering to a patient with a CSD associated disorder a calcitonin gene-related peptide antagonist (CGRP-antagonists), a clostridial derivative, or combination thereof. In some embodiments, the CSD-related symptoms are other than headache or migraine. In some embodiments, the CSD-associated disorder comprises migraine and migraine variants, stroke and other causes of brain ischemia, subarachnoid and intracranial hemorrhage, traumatic brain injury, seizures, head injury/head trauma (concussion), post traumatic stress disorder, fibromyalgia and tinnitus (either associated with or not associated with another disorder). In some embodiments, the CSD associated disorder is selected from stroke and other causes of brain ischemia, subarachnoid and intracranial hemorrhage, traumatic brain injury, seizures, and head injury/head trauma (concussion). In some embodiments, the CSD-associated symptoms are other than headache, migraine or migraine variants.

In another aspect, there is provided a method for preventing, alleviating or reducing the intensity or frequency of occurrence of a symptom related to or resulting from a CSD associated disorder, the method comprises administering to a patient with the CSD associated disorder a calcitonin gene-related peptide antagonist (CGRP-antagonists), a clostridial derivative, or combination thereof. In some embodiments, the administering is carried out between an initial time when the CSD associated disorder occurs and the onset of the resulting CSD associated symptom. In some embodiments, the administering is carried out after the occurrence of the CSD associated disorder and before the onset of the resulting CSD associated symptom. In some embodiments, the administration of the combination produces a synergistic effect relative to treatment or prevention with the CGRP antagonist or the clostridial derivative alone. In some embodiments, the CSD associated disorder is selected from stroke and other causes of brain ischemia, subarachnoid and intracranial hemorrhage, traumatic brain injury, seizures, head injury/head trauma (concussion), post-traumatic stress disorder after a head trauma and fibromyalgia. In some embodiments, the method comprises preventing, alleviating or reducing the intensity or frequency of occurrence of a CSD-related disorder and/or symptom in a patient in need thereof. In some embodiments, the CSD-related disorder is selected from stroke and other causes of brain ischemia, subarachnoid and intracranial hemorrhage, traumatic brain injury, seizures, head injury/head trauma (concussion), post-traumatic stress disorder after a head trauma and fibromyalgia.

In some embodiments, the CSD-related disorder is a traumatic brain injury. In some embodiments, the method comprises administering a CGRP-antagonist, a clostridial derivative or combination thereof between the time when the traumatic brain injury occurs and the onset of the TBI associated/related symptom. In some embodiments, the administering is carried out after the occurrence of the traumatic brain injury and before the onset of the TBI related symptom. In some embodiments, the administering is carried out after the traumatic brain injury and prior to onset of at least one of the traumatic brain injury associated symptoms. The method as disclosed herein prevents, reduces the intensity or occurrence of traumatic brain injury associated symptoms. Traumatic brain injury symptoms include hypotension, hypoxemia, brain swelling, headache, mental fog, decreased attention, decreased reaction time, neck pain, a difficulty remembering, a difficulty concentrating, a difficulty making decisions, fatigue, a mood change, nausea, sleep disturbance, mild motor disturbance, photophobia, blurred vision, ear ringing, a loss of sense of taste, and a loss of sense of smell, seizures, coma, muscle weakness, paralysis, memory loss, sexual dysfunction, cognitive impairment, post traumatic stress disorder, fibromyalgia and a progressive decline in neurologic function.

In some embodiments, the administering is carried out within four months of the traumatic brain injury. In some embodiments, the administering is carried out within 1 hour, 2 hours, 3 hours, 6 hours, 12 hours, 24 hours, 48 hours or 72 hours of the traumatic brain injury. In alternative embodiments, the administering is carried out within 1 week, two weeks, three weeks, four weeks, five weeks, six weeks, seven weeks, eight weeks, nine weeks, ten weeks, eleven weeks, twelve weeks, thirteen weeks, fourteen weeks, fifteen weeks, sixteen weeks or up to twenty four weeks of the traumatic brain injury. In some embodiments, the administering is carried out before the onset of at least one of the traumatic brain injury associated symptoms.

In alternative embodiments, the administering is carried out after the onset of a first traumatic brain injury associated symptom. In some embodiments, the administering is carried out within 1 hour, 2 hours, 3 hours, 6 hours, 12 hours, 24 hours, 48 hours or 72 hours of the onset of the first traumatic brain injury associated symptom. In alternative embodiments, the administering is carried out within 1 week, two weeks, three weeks, four weeks, five weeks, six weeks, seven weeks or eight weeks of the onset of the first traumatic brain injury associated symptom. In some embodiments, the first traumatic brain injury associated symptom is headache, mental fogginess, emotional disturbance, or combination thereof. In some embodiments, the administration of the combination produces a synergistic effect relative to treatment with the CGRP antagonist or the clostridial derivative alone.

In some embodiments, the clostridial derivative is a botulinum toxin. In one embodiment, the clostridial derivative is onabotulinumtoxinA. In some other embodiments, the clostridial derivative is a TEM. In some embodiments, the clostridial derivative is administered to a muscle, a nerve, a suture line, or combination thereof. In some embodiments, the administration can be by injection, for example, intramuscularly, intradermally and/or subcutaneously. In some embodiments, a TEM is administered in a dose sufficient to provide an intended therapeutic effect. In some embodiments, a therapeutically effective amount in the dose range of about 0.1 to 1000 μg of a TEM is administered.

In some embodiments, the clostridial derivative, for example a botulinum toxin, is administered to a trigeminal nerve. Trigeminal nerve administration of botulinum toxins has been disclosed for example in U.S. Pat. Nos. 8,609,112; 8,609,113; 8,734,810; 8,717,572; 9,238,061 and 10,064,921; each of which is hereby incorporated by reference in its entirety.

In some embodiments, the clostridial derivative, for example a botulinum toxin, is administered to a suture line. Suture line administration of botulinum toxins has been disclosed for example in U.S. Pat. Nos. 8,617,571; 9,248,168; 9,827,297; and 10,220,079; each of which is hereby incorporated by reference in its entirety.

In some embodiments, the methods comprise administering to a patient with a CSD related disorder a clostridial derivative.

In some embodiments, the CGRP-antagonist is an anti-calcitonin gene-related peptide receptor antibody (anti-CGRP antibody) or antigen-binding fragment thereof. For example, the antibody can be selected from galcanezumab, fremanezumab, eptinezumab or erenumab. In some embodiments, the anti-CGRP antibody or fragment thereof is administered at a dosage that is about 20% or 30% or 40% or 50% or 60% or 70% or 80% lower than the recommended dosage for the anti-CGRP antibody monotherapy. In some embodiments, the anti-CGRP antibody or antigen-binding fragment thereof is administered to a peripheral nerve, a cranial nerve, or combinations thereof.

For example, erenumab can be administered weekly, biweekly, monthly, every two months, every three months, every four months, every five months or every six months at a dosage of about 5 mg to about 500 mg.

Erenumab can be administered parenterally, subcutaneously or by peripheral administration. (Brauser D., Phase 3 STRIVE and ARISE Trials Show Efficacy, Safety for Erenumab in Migraine Prevention, Medscape Medical News, 2017)

In some embodiments, erenumab can be administered to the patient over the course of a set treatment period. (U.S. Patent Publication No. 20160311913) The treatment period can begin upon administration of a first dose erenumab and ends upon administration of a final dose of erenumab. The combination therapy with a clostridial derivative, for example a botulinum toxin, includes administration of botulinum toxin prior to, during or after the treatment period with erenumab. The treatment period may comprise from about 1 month to about 36 months, such as about 2 months, about 3 months, about 4 months, about 5 months, about 6 months, about 7 months, about 8 months, about 9 months, about 10 months, about 11 months, about 12 months, about 13 months, about 14 months, about 15 months, about 18 months, about 21 months, about 24 months, about 27 months, about 30 months, or about 33 months. In some embodiments, the treatment period is about 6 months. In other embodiments, the treatment period is about 7 months. In yet other embodiments, the treatment period is about 12 months. In certain embodiments, the treatment period can be longer than 36 months, such as 48 or 60 or 64 months or more. In some embodiment, erenumab is administered in a pharmaceutical composition comprising a buffer (preferably an acetate buffer), a surfactant (preferably polysorbate 20 or polysorbate 80), and a stabilizing agent (preferably sucrose). In one particular embodiment, the treatment period is at least about 6 months and produces a statistically significant reduction in the frequency, duration, or severity of chronic pain in the patient as compared to patients treated with erenumab or botulinum toxin alone.

In one embodiment, erenumab can be administered subcutaneously at a dose of about 5 mg to about 500 mg every one, two, three, four, five, six, seven, eight, nine or ten weeks.

In one embodiment, erenumab can be administered subcutaneously at a dose of about 10 mg to about 200 mg every one, two, three, four, five, six, seven, eight, nine or ten weeks.

In one embodiment, erenumab can be administered subcutaneously at a dose of about 25 mg to about 150 mg every one, two, three, four, five, six, seven, eight, nine or ten weeks.

In one embodiment, erenumab can be administered subcutaneously at a dose of about 90 mg to about 120 mg every one, two, three, four, five, six, seven, eight, nine or ten weeks.

In one embodiment, erenumab can be administered subcutaneously at a dose of about 50 mg to about 60 mg every one, two, three, four, five, six, seven, eight, nine or ten weeks.

In one embodiment, erenumab can be administered subcutaneously at a dose of about 70 mg every one, two, three, four, five, six, seven, eight, nine or ten weeks.

In one embodiment, erenumab can be administered subcutaneously at a dose of about 140 mg every one, two, three, four, five, six, seven, eight, nine or ten weeks.

In one embodiment, erenumab can be administered subcutaneously at a monthly dose of about 140 mg.

In one embodiment, erenumab can be administered subcutaneously at a monthly dose of about 70 mg.

In one embodiment, erenumab can be administered subcutaneously at a dose of about 140 mg every two months.

In one embodiment, erenumab can be administered subcutaneously at a dose of about 70 mg every two months.

In one embodiment, erenumab can be administered subcutaneously at a dose of about 140 mg every three months.

In one embodiment, erenumab can be administered subcutaneously at a dose of about 70 mg every three months.

In one embodiment, an anti-CGRP antibody galcanezumab can be administered weekly, biweekly, monthly, every two months, every three months, every four months, every five months or every six months at a dosage of about 5 mg to about 500 mg.

In some embodiments, galcanezumab can be administered to the patient over the course of a set treatment period. The treatment period can begin upon administration of a first dose galcanezumab and ends upon administration of a final dose of galcanezumab. The combination therapy with a clostridial derivative, for example a botulinum toxin, includes administration of botulinum toxin prior to, during or after the treatment period with galcanezumab. The treatment period may comprise from about 1 month to about 36 months, such as about 2 months, about 3 months, about 4 months, about 5 months, about 6 months, about 7 months, about 8 months, about 9 months, about 10 months, about 11 months, about 12 months, about 13 months, about 14 months, about 15 months, about 18 months, about 21 months, about 24 months, about 27 months, about 30 months, or about 33 months. In some embodiments, the treatment period is about 6 months. In other embodiments, the treatment period is about 7 months. In yet other embodiments, the treatment period is about 12 months. In certain embodiments, the treatment period can be longer than 36 months, such as 48 or 60 or 64 months or more. In one particular embodiment, the treatment period is at least about 6 months and produces a statistically significant reduction in the frequency, duration, or severity of pain in the patient as compared to patients treated with galcanezumab or botulinum toxin alone.

In one embodiment, galcanezumab is administered subcutaneously at a dose of about 10 mg to about 500 mg every one, two, three, four, five, six, seven, eight, nine or ten weeks.

In one embodiment, galcanezumab is administered subcutaneously at a dose of about 50 mg to about 300 mg every one, two, three, four, five, six, seven, eight, nine or ten weeks.

In one embodiment, galcanezumab is administered subcutaneously at a dose of about 75 mg to about 250 mg every one, two, three, four, five, six, seven, eight, nine or ten weeks.

In one embodiment, galcanezumab is administered subcutaneously at a dose of about 75 mg to about 100 mg every one, two, three, four, five, six, seven, eight, nine or ten weeks.

In one embodiment, galcanezumab is administered subcutaneously at a dose of about 150 mg to about 220 mg every one, two, three, four, five, six, seven, eight, nine or ten weeks.

In one embodiment, galcanezumab is administered subcutaneously at a dose of about 120 mg every one, two, three, four, five, six, seven, eight, nine or ten weeks.

In one embodiment, galcanezumab is administered subcutaneously at a dose of about 240 mg every one, two, three, four, five, six, seven, eight, nine or ten weeks.

In one embodiment, galcanezumab is administered subcutaneously at a monthly dose of about 240 mg.

In one embodiment, galcanezumab is administered subcutaneously at a monthly dose of about 120 mg.

In one embodiment, galcanezumab is administered subcutaneously at a dose of about 240 mg every two months.

In one embodiment, galcanezumab is administered subcutaneously at a dose of about 120 mg every two months.

In one embodiment, galcanezumab is administered subcutaneously at a dose of about 240 mg every three months.

In one embodiment, galcanezumab is administered subcutaneously at a dose of about 120 mg every three months.

In some embodiments, fremanezumab can be administered to the patient over the course of a set treatment period. (Silberstein, S. D., et. al., N Engl J Med 2017; 377:2113-22.) The treatment period can begin upon administration of a first dose fremanezumab and ends upon administration of a final dose of fremanezumab. The combination therapy with a clostridial derivative, for example a botulinum toxin, includes administration of botulinum toxin prior to, during or after the treatment period with fremanezumab. The treatment period may comprise from about 1 month to about 36 months, such as about 2 months, about 3 months, about 4 months, about 5 months, about 6 months, about 7 months, about 8 months, about 9 months, about 10 months, about 11 months, about 12 months, about 13 months, about 14 months, about 15 months, about 18 months, about 21 months, about 24 months, about 27 months, about 30 months, or about 33 months. In some embodiments, the treatment period is about 6 months. In other embodiments, the treatment period is about 7 months. In yet other embodiments, the treatment period is about 12 months. In certain embodiments, the treatment period can be longer than 36 months, such as 48 or 60 or 64 months or more. In one particular embodiment, the treatment period is at least about 6 months and produces a statistically significant reduction in the frequency, duration, or severity of pain in the patient as compared to patients treated with fremanezumab or botulinum toxin alone.

In one embodiment, fremanezumab is administered subcutaneously at a dose of about 100 mg to about 1000 mg every one, two, three, four, five, six, seven, eight, nine or ten weeks.

In one embodiment, fremanezumab is administered subcutaneously at a dose of about 150 mg to about 700 mg every one, two, three, four, five, six, seven, eight, nine or ten weeks.

In one embodiment, fremanezumab is administered subcutaneously at a dose of about 150 mg to about 500 mg every one, two, three, four, five, six, seven, eight, nine or ten weeks.

In one embodiment, fremanezumab is administered subcutaneously at a dose of about 150 mg to about 200 mg every one, two, three, four, five, six, seven, eight, nine or ten weeks.

In one embodiment, fremanezumab is administered subcutaneously at a dose of about 150 mg to about 500 mg every one, two, three, four, five, six, seven, eight, nine or ten weeks.

In one embodiment, fremanezumab is administered subcutaneously at a dose of about 225 mg every one, two, three, four, five, six, seven, eight, nine or ten weeks.

In one embodiment, fremanezumab is administered subcutaneously at a dose of about 450 mg every one, two, three, four, five, six, seven, eight, nine or ten weeks.

In one embodiment, fremanezumab is administered subcutaneously at a dose of about 675 mg every one, two, three, four, five, six, seven, eight, nine or ten weeks.

In one embodiment, fremanezumab is administered subcutaneously at a monthly dose of about 225 mg.

In one embodiment, fremanezumab is administered subcutaneously at a monthly dose of about 450 mg.

In one embodiment, fremanezumab is administered subcutaneously at a monthly dose of about 675 mg.

In one embodiment, fremanezumab is administered subcutaneously at a dose of about 225 mg every two months.

In one embodiment, fremanezumab is administered subcutaneously at a dose of about 450 mg every two months.

In one embodiment, fremanezumab is administered subcutaneously at a dose of about 225 mg every three months.

In one embodiment, fremanezumab is administered subcutaneously at a dose of about 450 mg every three months.

In one embodiment, fremanezumab is administered subcutaneously at a dose of about 675 mg every three months.

In some embodiments, eptinezumab can be administered to the patient over the course of a set treatment period. The treatment period can begin upon administration of a first dose eptinezumab and ends upon administration of a final dose of eptinezumab. The combination therapy with a clostridial derivative, for example a botulinum toxin, includes administration of botulinum toxin prior to, during or after the treatment period with eptinezumab. The treatment period may comprise from about 1 month to about 36 months, such as about 2 months, about 3 months, about 4 months, about 5 months, about 6 months, about 7 months, about 8 months, about 9 months, about 10 months, about 11 months, about 12 months, about 13 months, about 14 months, about 15 months, about 18 months, about 21 months, about 24 months, about 27 months, about 30 months, or about 33 months. In some embodiments, the treatment period is about 6 months. In other embodiments, the treatment period is about 7 months. In yet other embodiments, the treatment period is about 12 months. In certain embodiments, the treatment period can be longer than 36 months, such as 48 or 60 or 64 months or more. In one particular embodiment, the treatment period is at least about 6 months and produces a statistically significant reduction in the frequency, duration, or severity of pain in the patient as compared to patients treated with eptinezumab or botulinum toxin alone.

In one embodiment, eptinezumab is administered subcutaneously at a dose of about 50 mg to about 1000 mg every one, two, three, four, five, six, seven, eight, nine or ten weeks.

In one embodiment, eptinezumab is administered subcutaneously at a dose of about 100 mg to about 700 mg every one, two, three, four, five, six, seven, eight, nine or ten weeks.

In one embodiment, eptinezumab is administered subcutaneously at a dose of about 200 mg to about 500 mg every one, two, three, four, five, six, seven, eight, nine or ten weeks.

In one embodiment, eptinezumab is administered subcutaneously at a dose of about 250 mg to about 350 mg every one, two, three, four, five, six, seven, eight, nine or ten weeks.

In one embodiment, eptinezumab is administered subcutaneously at a dose of about 300 mg every one, two, three, four, five, six, seven, eight, nine or ten weeks.

In one embodiment, eptinezumab is administered subcutaneously at a monthly dose of about 100 mg.

In one embodiment, eptinezumab is administered subcutaneously at a monthly dose of about 200 mg.

In one embodiment, eptinezumab is administered subcutaneously at a monthly dose of about 300 mg.

In one embodiment, eptinezumab is administered subcutaneously at a dose of about 100 mg every two months.

In one embodiment, eptinezumab is administered subcutaneously at a dose of about 200 mg every two months.

In one embodiment, eptinezumab is administered subcutaneously at a dose of about 300 mg every two months.

In one embodiment, eptinezumab is administered subcutaneously at a dose of about 100 mg every three months.

In one embodiment, eptinezumab is administered subcutaneously at a dose of about 200 mg every three months.

In one embodiment, eptinezumab is administered subcutaneously at a dose of about 300 mg every three months.

In some embodiments, an antagonist of CGRP receptor can be administered in combination with a clostridial derivative. Preferably, the CGRP antagonist is selected from ubrogepant, atogepant, rimegepant or a pharmaceutically acceptable salt thereof.

In some embodiments, ubrogepant can be administered to the patient over the course of a set treatment period or indefinitely. The treatment period can begin upon administration of a first dose ubrogepant and continue until the patient is administered ubrogepant on a regular or intermittent basis. The combination therapy with a clostridial derivative, for example a botulinum toxin, includes administration of botulinum toxin prior to, during or after the treatment period with ubrogepant. The treatment period may comprise from about 1 month to about 36 months, such as about 2 months, about 3 months, about 4 months, about 5 months, about 6 months, about 7 months, about 8 months, about 9 months, about 10 months, about 11 months, about 12 months, about 13 months, about 14 months, about 15 months, about 18 months, about 21 months, about 24 months, about 27 months, about 30 months, or about 33 months. In some embodiments, the treatment period is about 6 months. In other embodiments, the treatment period is about 7 months. In yet other embodiments, the treatment period is about 12 months. In certain embodiments, the treatment period can be longer than 36 months, such as 48 or 60 or 64 months or more. In one particular embodiment, the treatment period is at least about 6 months and produces a statistically significant reduction in the frequency, duration, or severity of pain in the patient as compared to patients treated with ubrogepant or botulinum toxin alone.

In some embodiments, ubrogepant is administered at an oral dose of about 5 to about 500 mg once, twice or three times a day.

In some embodiments, ubrogepant is administered at an oral dose of about 25 mg once, twice or three times a day.

In some embodiments, ubrogepant is administered at an oral dose of about 50 mg once, twice or three times a day.

In some embodiments, ubrogepant is administered at an oral dose of about 100 mg once, twice or three times a day.

In some embodiments, ubrogepant is administered at an oral dose of about 200 mg once, twice or three times a day.

In some embodiments, atogepant can be administered to the patient over the course of a set treatment period or indefinitely. The treatment period can begin upon administration of a first dose atogepant and continue until the patient is administered atogepant on a regular or intermittent basis. The combination therapy with a clostridial derivative, for example a botulinum toxin, includes administration of botulinum toxin prior to, during or after the treatment period with atogepant. The treatment period may comprise from about 1 month to about 36 months, such as about 2 months, about 3 months, about 4 months, about 5 months, about 6 months, about 7 months, about 8 months, about 9 months, about 10 months, about 11 months, about 12 months, about 13 months, about 14 months, about 15 months, about 18 months, about 21 months, about 24 months, about 27 months, about 30 months, or about 33 months. In some embodiments, the treatment period is about 6 months. In other embodiments, the treatment period is about 7 months. In yet other embodiments, the treatment period is about 12 months. In certain embodiments, the treatment period can be longer than 36 months, such as 48 or 60 or 64 months or more. In one particular embodiment, the treatment period is at least about 6 months and produces a statistically significant reduction in the frequency, duration, or severity of pain in the patient as compared to patients treated with atogepant or botulinum toxin alone.

In some embodiments, atogepant is administered at an oral dose of about 5 to about 500 mg once, twice or three times a day.

In some embodiments, atogepant is administered at an oral dose of about 25 mg once, twice or three times a day.

In some embodiments, atogepant is administered at an oral dose of about 50 mg once, twice or three times a day.

In some embodiments, atogepant is administered at an oral dose of about 100 mg once, twice or three times a day.

In some embodiments, atogepant is administered at an oral dose of about 200 mg once, twice or three times a day.

In some embodiments, rimegepant can be administered to the patient over the course of a set treatment period or indefinitely. The treatment period can begin upon administration of a first dose rimegepant and continue until the patient is administered rimegepant on a regular or intermittent basis. The combination therapy with botulinum toxin includes administration of botulinum toxin prior to, during or after the treatment period with rimegepant. The treatment period may comprise from about 1 month to about 36 months, such as about 2 months, about 3 months, about 4 months, about 5 months, about 6 months, about 7 months, about 8 months, about 9 months, about 10 months, about 11 months, about 12 months, about 13 months, about 14 months, about 15 months, about 18 months, about 21 months, about 24 months, about 27 months, about 30 months, or about 33 months. In some embodiments, the treatment period is about 6 months. In other embodiments, the treatment period is about 7 months. In yet other embodiments, the treatment period is about 12 months. In certain embodiments, the treatment period can be longer than 36 months, such as 48 or 60 or 64 months or more. In one particular embodiment, the treatment period is at least about 6 months and produces a statistically significant reduction in the frequency, duration, or severity of pain in the patient as compared to patients treated with rimegepant or botulinum toxin alone.

In some embodiments, rimegepant is administered at an oral dose of about 5 to about 500 mg once, twice or three times a day.

In some embodiments, rimegepant is administered at an oral dose of about 25 mg once, twice or three times a day.

In some embodiments, rimegepant is administered at an oral dose of about 50 mg once, twice or three times a day.

In some embodiments, rimegepant is administered at an oral dose of about 100 mg once, twice or three times a day.

In some embodiments, rimegepant is administered at an oral dose of about 200 mg once, twice or three times a day.

In some embodiments, rimegepant is administered at an oral dose of about 5 to about 500 mg once, twice or three times a day.

In some embodiments, rimegepant is administered at an oral dose of about 25 mg once, twice or three times a day.

In some embodiments, rimegepant is administered at an oral dose of about 50 mg once, twice or three times a day.

In some embodiments, rimegepant is administered at an oral dose of about 100 mg once, twice or three times a day.

In some embodiments, rimegepant is administered at an oral dose of about 200 mg once, twice or three times a day.

In some embodiments, the administration of a clostridial derivative, a CGRP antagonist, or combination thereof reduces the frequency, severity and/or duration of pain in patients in need thereof.

In some embodiments, the present method described herein is administered to a patient undergoing treatment with one or more additional medications for the treatment of pain. For example, morphine, codeine, hydrocodone, oxycodone, fentanyl, pethidine, methadone, pentazocine, sufentanil, levorphanol, dihydrocodeine, nalbuphine, butorphanol, tramadol, meptazinol, buprenorphine, dipipanone, alfentanil, remifentanil, oxymorphone, tapentadol, propoxyphene or hydromorphone. In one embodiment, the additional medication is acetaminophen, ibuprofen, ketaprofen, naproxen or aspirin. In one embodiment, the additional medication is a treatment for arthritis, for example, infliximab (Remicade), adalimumab (Humira), certolizumab pegol (Cimzia), golimumab (Simponi), or etanercept.

In some embodiments, the clostridial derivative is a botulinum toxin. In some embodiments, the clostridial derivative is onabotulinumtoxinA and is administered at a dose of about 1 unit, about 2 units, about 3 units, about 4 units, about 5 units, about 6 units, about 7 units, about 8 units, about 9 units or about 10 units. The frequency of administration can be once every one, two, three, four, five, six, seven, eight, nine, ten, eleven, twelve, thirteen, fourteen, fifteen or sixteen weeks.

In some embodiments, onabotulinumtoxinA is administered at a dose of about 10 unit, about 15 units, about 20 units, about 25 units, about 30 units, about 40 units, about 45 units, about 50 units, about 55 units or about 60 units. The frequency of administration can be once every one, two, three, four, five, six, seven, eight, nine, ten, eleven, twelve, thirteen, fourteen, fifteen or sixteen weeks.

In some embodiments, the clostridial derivative is onabotulinumtoxinA and is administered at a dose of about of about 25 unit, about 50 units, about 75 units, about 100 units, about 125 units, about 150 units, about 175 units, about 200 units, about 225 units or about 250 units every one, two, three, four, five, six, seven, eight, nine, ten, eleven, twelve, thirteen, fourteen, fifteen or sixteen weeks.

In some embodiments, the clostridial derivative is onabotulinumtoxinA and is administered at a dose of about dose of about 1 to about 1,000 units. The frequency of administration can be once every one, two, three, four, five, six, seven, eight, nine, ten, eleven, twelve, thirteen, fourteen, fifteen or sixteen weeks.

In some embodiments, the clostridial derivative is onabotulinumtoxinA and is administered at a dose of about 1 to about 100 units every one, two, three, four, five, six, seven, eight, nine, ten, eleven, twelve, thirteen, fourteen, fifteen or sixteen weeks.

In some embodiments, the clostridial derivative is onabotulinumtoxinA and is administered at a dose of about 10 to about 50 units. The frequency of administration can be once every one, two, three, four, five, six, seven, eight, nine, ten, eleven, twelve, thirteen, fourteen, fifteen or sixteen weeks.

In some embodiments, onabotulinumtoxinA is administered peripherally.

In some embodiments, onabotulinumtoxinA is administered parenterally.

In some embodiments, onabotulinumtoxinA is administered topically.

In some embodiments, onabotulinumtoxinA is administered subcutaneously or intramuscularly.

In some embodiments, onabotulinumtoxinA is administered subcutaneously once every month or two months.

In some embodiments, onabotulinumtoxinA is administered at a dose of about 155 units.

The effective amount of the clostridial derivative can be measured in mass units (e.g. in ng or mg). The effective dose in weight units can be determined based on the intended effect. For example, the effective weight can be determined based on the amount of clostridial derivative required to have a therapeutic effect on the muscle or a sensory effect. In some embodiments, the clostridial derivative can be administered at a dose of about 0.001 ng to about 1000 ng, preferably about 0.001 ng to about 500 ng, preferably about 0.01 ng to about 250 ng, preferably about 0.1 ng to about 150 ng, preferably about 1 ng to about 100 ng, preferably about 1 ng to about 10 ng. For example, onabotulinumtoxinA can be administered at a dose of about 1 ng, 2 ng, 3 ng, 4 ng, 5 ng, 6 ng, 7 ng, 8 ng, 9 ng or 10 ng.

In one embodiment, the CGRP-antagonist can be administered intravenously. The intravenous formulation can contain a tonicity modifier to avoid crenation or hemolysis of red blood cells, and/or to mitigate or avoid pain and discomfort to the patient. Preferably, the formulation to be administered to the patient has an effective osmotic pressure that is approximately the same as that of the blood of the patient. Tonicity modifiers can be non-ionic tonicity modifiers such as glycerol, sorbitol, mannitol, sucrose, propylene glycol or dextrose, or a mixture thereof. Preferably the non-ionic tonicity modifier is dextrose, sucrose or mannitol, or a mixture thereof. Aqueous pharmaceutical formulations for intravenous administration generally can have a pH of from 3 to 9.

In some embodiments, the CGRP-antagonist can be administered orally, sublingually, transdermally, subcutaneously, intravenously, or intramuscularly.

Stable liquid or solid pharmaceutical composition comprising a clostridical toxin derivative, a disaccharide, a surfactant and an antioxidant can be used in combination with CGRP-antagonists.

CGRP is a member of the calcitonin family of peptides, which in human exists in two form, α-CGRP and β-CGRP. α-CGRP and β-CGRP vary by three amino acids, have similar activities and exhibit differential distribution. At least two CGRP receptor subtypes may also account for differential activities. CGRP is produced in both peripheral and central neurons, and released by the trigeminal nerve. CGRP has been shown to be a potent vasodilator in the periphery, where CGRP-containing neurons are closely associated with blood vessels.

CGRP is released by sensory nerves, e.g. the trigeminal nerve, which innervates part of the skin of the face. The trigeminal nerve has three major branches, a number of smaller branches and is the great sensory nerve of the head and neck, carrying touch, temperature, pain, and proprioception (position sense) signals from the face and scalp to the brainstem. Trigeminal sensory fibers originate in the skin, course toward the trigeminal ganglion (a sensory nerve cell body), pass through the trigeminal ganglion, and travel within the trigeminal nerve to the sensory nucleus of the trigeminal nerve located in the brainstem.

### Definitions

As used herein, the words or terms set forth below have the following definitions:

“About” or “approximately” as used herein means within an acceptable error range for the particular value as determined by one of ordinary skill in the art, which will depend in part on how the value is measured or determined, (i.e., the limitations of the measurement system). For example, “about” can mean within 1 or more than 1 standard deviations, per practice in the art. Where particular values are described in the application and claims, unless otherwise stated, the term “about” means within an acceptable error range for the particular value.

“Administration”, or “to administer” means the step of giving (i.e. administering) a pharmaceutical composition to a subject, or alternatively a subject receiving a pharmaceutical composition. The CGRP antagonist, clostridial derivative, or combination thereof can be administered intravenously, peripherally, extracranially, orally, intramuscularly, subdermally, intradermally or topically.

“Alleviating” means a reduction in the occurrence of a pain, or of any symptom or cause of a condition or disorder. Thus, alleviating includes some reduction, significant reduction, near total reduction, and total reduction.

“Biological activity” describes the beneficial or adverse effects of a drug on living matter. When a drug is a complex chemical mixture, this activity is exerted by the substance's active ingredient but can be modified by the other constituents. Biological activity of a clostridial derivative such as a botulinum toxin can be assessed as potency or as toxicity by an in vivo LD₅₀ or ED₅₀ assay, or through an in vitro assay such as, for example, cell-based potency assays as described in U.S. 20100203559 and U.S. 20100233802.

“Botulinum toxin” means a neurotoxin produced by Clostridium botulinum, as well as a botulinum toxin, fragments, variants or chimeras thereof made recombinantly by a non-Clostridial species. The phrase “botulinum toxin”, as used herein, encompasses Botulinum toxin serotype A (BoNT/A), Botulinum toxin serotype B (BoNT/B), Botulinum toxin serotype C (BoNT/C), Botulinum toxin serotype D (BoNT/D), Botulinum toxin serotype E (BoNT/E), Botulinum toxin serotype F (BoNT/F), Botulinum toxin serotype G (BoNT/G), Botulinum toxin serotype H (BoNT/H), Botulinum toxin serotype X (BoNT/X), Botulinum toxin serotype En (BoNT/En), and mosaic Botulinum toxins and/or their subtypes and any other types of subtypes thereof, or any re-engineered proteins, analogs, derivatives, homologs, parts, sub-parts, variants, or versions, in each case, of any of the foregoing. “Botulinum toxin”, as used herein, also encompasses a “modified botulinum toxin”. Further “botulinum toxin” as used herein also encompasses a botulinum toxin complex, (for example, the 300, 600 and 900 kDa complexes), as well as the neurotoxic component of the botulinum toxin (150 kDa) that is unassociated with the complex proteins.

“CGRP”, abbreviated for Calcitonin-Gene-Related-Peptide, as used herein encompasses any member of the calcitonin family, including any calcitonin gene related peptide and analogs, calcitonin, amylin, adrenomedullin and their analogs.

“CGRP antagonist” refers to any molecule that exhibits any one or more of the following characteristics: (a) bind to CGRP or CGRP-R and the binding results in a reduction or inhibition of CGRP activity; (b) block CGRP from binding to its receptor(s); (c) block or decrease CGRP receptor activation; (d) inhibit CGRP biological activity or downstream pathways mediated by CGRP signaling function; (e) increase clearance of CGRP; and (f) inhibit or reduce CGRP synthesis, production or release. CGRP antagonists include but are not limited to antibodies to CGRP, antibodies to the CGRP-R, small molecules that antagonize CGRP, and small molecules that antagonize CGRP-R.

“Clostridial derivative” refers to a molecule which contains any part of a clostridial toxin as defined herein. As used herein, the term “clostridial derivative” encompasses native or recombinant neurotoxins, recombinant modified toxins, fragments, chimeras and variants thereof, a Targeted Vesicular Exocytosis Modulator (TEM), or combinations thereof.

“Clostridial toxin” refers to any toxin produced by a Clostridial toxin strain that can execute the overall cellular mechanism whereby a Clostridial toxin intoxicates a cell and encompasses the binding of a Clostridial toxin to a low or high affinity Clostridial toxin receptor, the internalization of the toxin/receptor complex, the translocation of the Clostridial toxin light chain into the cytoplasm and the enzymatic modification of a Clostridial toxin substrate. Non-limiting examples of Clostridial toxins include a Botulinum toxin like BoNT/A, a BoNT/B, a BoNT/C₁, a BoNT/D, a BoNT/E, a BoNT/F, a BoNT/G, BoNT/H, a BoNT/En, BoNT/X, mosaic botulinum toxins, a Tetanus toxin (TeNT), a Baratii toxin (BaNT), and a Butyricum toxin (BuNT). The BoNT/C₂ cytotoxin and BoNT/C₃ cytotoxin, not being neurotoxins, are excluded from the term “Clostridial toxin.” A Clostridial toxin disclosed herein includes, without limitation, naturally occurring Clostridial toxin variants, such as, e.g., Clostridial toxin isoforms and Clostridial toxin subtypes; non-naturally occurring Clostridial toxin variants, such as, e.g., conservative Clostridial toxin variants, non-conservative Clostridial toxin variants, Clostridial toxin chimeric variants and active Clostridial toxin fragments thereof, or any combination thereof. A Clostridial toxin disclosed herein also includes a Clostridial toxin complex. As used herein, the term “Clostridial toxin complex” refers to a complex comprising a Clostridial toxin and non-toxin associated proteins (NAPs), such as, e.g., a Botulinum toxin complex, a Tetanus toxin complex, a Baratii toxin complex, and a Butyricum toxin complex. Non-limiting examples of Clostridial toxin complexes include those produced by a Clostridium botulinum, such as, e.g., a 900-kDa BoNT/A complex, a 500-kDa BoNT/A complex, a 300-kDa BoNT/A complex, a 500-kDa BoNT/B complex, a 500-kDa BoNT/C₁ complex, a 500-kDa BoNT/D complex, a 300-kDa BoNT/D complex, a 300-kDa BoNT/E complex, and a 300-kDa BoNT/F complex.

“Clostridial toxin active ingredient” refers to a molecule which contains any part of a clostridial toxin that exerts an effect upon or after administration to a subject or patient. As used herein, the term “clostridial toxin active ingredient” or “clostridial derivative” encompasses native or recombinant neurotoxins, recombinant modified toxins, fragments, chimeras and variants thereof, a Targeted vesicular Exocytosis Modulator (TEM), or combinations thereof.

“Combination therapy” refers to a treatment wherein a clostridial derivative, for example a botulinum toxin, and a CGRP antagonist are administered either simultaneously or sequentially, by a similar administration route or by different administration routes.

“Effective amount” as applied to the biologically active ingredient means that amount of the ingredient which is generally sufficient to effect a desired change in the subject. For example, where the desired effect is a reduction in occurrence of a CSD related disorder, an effective amount of the ingredient is that amount which causes at least a substantial reduction in occurrence of the CSD related disorder, and without resulting in significant toxicity.

“Extracranial administration” means administration to a site external to the cranium. In some embodiments, extracranial administration refers to administration to any suture line, or combination of suture lines on the skull of a patient.

“Implant” means a controlled release (e.g., pulsatile or continuous) composition or drug delivery system. The implant can be, for example, injected, inserted or implanted into a human body.

“Intramuscular” or “intramuscularly” means into or within (as in administration or injection of a CGRP antagonist into) a muscle.

“Local administration” means direct administration of a pharmaceutical at or to the vicinity of a site on or within an animal body, at which site a biological effect of the pharmaceutical is desired, such as via, for example, intramuscular or intra- or subdermal injection or topical administration. Topical administration is a type of local administration in which a pharmaceutical agent is applied to a patient's skin.

“Modified botulinum toxin” means a botulinum toxin that has had at least one of its amino acids deleted, modified, or replaced, as compared to a native botulinum toxin. Additionally, the modified botulinum toxin can be a recombinantly produced neurotoxin, or a derivative or fragment of a recombinantly made neurotoxin. A modified botulinum toxin retains at least one biological activity of the native botulinum toxin, such as, the ability to bind to a botulinum toxin receptor, or the ability to inhibit neurotransmitter release from a neuron. One example of a modified botulinum toxin is a botulinum toxin that has a light chain from one botulinum toxin serotype (such as serotype A), and a heavy chain from a different botulinum toxin serotype (such as serotype B). Another example of a modified botulinum toxin is a botulinum toxin coupled to a neurotransmitter, such as substance P.

“Mutation” means a structural modification of a naturally occurring protein or nucleic acid sequence. For example, in the case of nucleic acid mutations, a mutation can be a deletion, addition or substitution of one or more nucleotides in the DNA sequence. In the case of a protein sequence mutation, the mutation can be a deletion, addition or substitution of one or more amino acids in a protein sequence. For example, a specific amino acid comprising a protein sequence can be substituted for another amino acid, for example, an amino acid selected from a group which includes the amino acids alanine, aspargine, cysteine, aspartic acid, glutamic acid, phenylalanine, glycine, histidine, isoleucine, lysine, leucine, methionine, proline, glutamine, arginine, serine, threonine, valine, tryptophan, tyrosine or any other natural or non-naturally occurring amino acid or chemically modified amino acids. Mutations to a protein sequence can be the result of mutations to DNA sequences that when transcribed, and the resulting mRNA translated, produce the mutated protein sequence. Mutations to a protein sequence can also be created by fusing a peptide sequence containing the desired mutation to a desired protein sequence.

“Patient” means a human or non-human subject receiving medical or veterinary care.

“Peripherally administering” or “peripheral administration” means administration by means of a peripheral location on a patient. Peripheral administration includes subdermal, intradermal, transdermal, subcutaneous administration, and intramuscular administration as well as extra-axial (outside of the bony structures) and extradural (outside of the dura including outside of the skull and spinal column).

“Pharmaceutical composition” means a composition comprising an active pharmaceutical ingredient, such as, for example, a clostridial toxin active ingredient such as a botulinum toxin, and at least one additional ingredient, such as, for example, a stabilizer or excipient or the like. A pharmaceutical composition is therefore a formulation which is suitable for diagnostic or therapeutic administration to a subject, such as a human patient. The pharmaceutical composition can be, for example, in a lyophilized or vacuum dried condition, a solution formed after reconstitution of the lyophilized or vacuum dried pharmaceutical composition, or as a solution or solid which does not require reconstitution.

“Pharmacologically acceptable excipient” is synonymous with “pharmacological excipient” or “excipient” and refers to any excipient that has substantially no long term or permanent detrimental effect when administered to mammal and encompasses compounds such as, e.g., stabilizing agent, a bulking agent, a cryo-protectant, a lyo-protectant, an additive, a vehicle, a carrier, a diluent, or an auxiliary. An excipient generally is mixed with an active ingredient, or permitted to dilute or enclose the active ingredient and can be a solid, semi-solid, or liquid agent. It is also envisioned that a pharmaceutical composition comprising a Clostridial toxin active ingredient can include one or more pharmaceutically acceptable excipients that facilitate processing of an active ingredient into pharmaceutically acceptable compositions. Insofar as any pharmacologically acceptable excipient is not incompatible with the Clostridial toxin active ingredient, its use in pharmaceutically acceptable compositions is contemplated. Non-limiting examples of pharmacologically acceptable excipients can be found in, e.g., Pharmaceutical Dosage Forms and Drug Delivery Systems (Howard C. Ansel et al., eds., Lippincott Williams & Wilkins Publishers, 7^{th} ed. 1999); Remington: The Science and Practice of Pharmacy (Alfonso R. Gennaro ed., Lippincott, Williams & Wilkins, 20^{th} ed. 2000); Goodman & Gilman's The Pharmacological Basis of Therapeutics (Joel G. Hardman et al., eds., McGraw-Hill Professional, 10^{th} ed. 2001); and Handbook of Pharmaceutical Excipients (Raymond C. Rowe et al., APhA Publications, 4^{th} edition 2003), each of which is hereby incorporated by reference in its entirety.

“Stabilizing agent”, “stabilization agent” or “stabilizer” means a substance that acts to stabilize a Clostridial toxin active ingredient.

“Stabilizers” can include excipients and can include protein and non-protein molecules.

“TEM” as used herein, is synonymous with “Targeted Exocytosis Modulator” or “retargeted endopeptidase.” Generally, a TEM comprises an enzymatic domain from a Clostridial toxin light chain, a translocation domain from a Clostridial toxin heavy chain, and a targeting domain. The targeting domain of a TEM provides an altered cell targeting capability that targets the molecule to a receptor other than the native Clostridial toxin receptor utilized by a naturally-occurring Clostridial toxin. This re-targeted capability is achieved by replacing the naturally-occurring binding domain of a Clostridial toxin with a targeting domain having a binding activity for a non-Clostridial toxin receptor. Although binding to a non-Clostridial toxin receptor, a TEM undergoes all the other steps of the intoxication process including internalization of the TEM/receptor complex into the cytoplasm, formation of the pore in the vesicle membrane and di-chain molecule, translocation of the enzymatic domain into the cytoplasm, and exerting a proteolytic effect on a component of the SNARE complex of the target cell.

“Therapeutic formulation” means a formulation can be used to treat and thereby alleviate a disorder or a disease, such as, for example, a CSD related disorder.

“Topical administration” excludes systemic administration of the neurotoxin. In other words, and unlike conventional therapeutic transdermal methods, topical administration of botulinum toxin does not result in significant amounts, such as the majority of, the neurotoxin passing into the circulatory system of the patient.

“Treating” or “treatment” means to alleviate (or to eliminate) a CSD-related condition or disorder and/or symptoms thereof, either temporarily or permanently. More particularly, as used herein “treatment” of a CSD-related condition and/or symptoms thereof includes one or more of reducing the frequency (or to prevent) or reducing the duration of the CSD-related condition and/or at least one symptom thereof, ameliorating the severity of the CSD-related condition and/or at least one symptom thereof, treating the CSD-related condition and/or at least one symptom thereof, curing the CSD-related condition and/or at least one symptom thereof, achieving a reduction in the number or severity of the CSD-related condition and/or at least one symptom thereof, improving the quality of life of the patient experiencing the CSD-related condition and/or at least one symptom thereof.

“Variant” means a clostridial neurotoxin, such as wild-type botulinum toxin serotype A, B, C, D, E, F, G, H, X, En or mosaic botulinum toxins that has been modified by the replacement, modification, addition or deletion of at least one amino acid relative to wild-type botulinum toxin, which is recognized by a target cell, internalized by the target cell, and catalytically cleaves a SNARE (SNAP (Soluble NSF Attachment Protein) Receptor) protein in the target cell.

In some embodiments, the clostridial derivative of the present method includes a native, recombinant clostridial toxin, recombinant modified toxin, fragments thereof, targeted exocytosis modulators (TEMs), or combinations thereof. In some embodiments, the clostridial derivative is a botulinum toxin. In alternative embodiments, the clostridial derivative is a TEM.

In some embodiments, the botulinum neurotoxin can be a modified neurotoxin, that is a botulinum neurotoxin which has at least one of its amino acids deleted, modified or replaced, as compared to a native toxin, or the modified botulinum neurotoxin can be a recombinant produced botulinum neurotoxin or a derivative or fragment thereof. In certain embodiments, the modified toxin has an altered cell targeting capability for a neuronal or non-neuronal cell of interest. This altered capability is achieved by replacing the naturally-occurring targeting domain of a botulinum toxin with a targeting domain showing a selective binding activity for a non-botulinum toxin receptor present in a non-botulinum toxin target cell. Such modifications to a targeting domain result in a modified toxin that is able to selectively bind to a non-botulinum toxin receptor (target receptor) present on a non-botulinum toxin target cell (re-targeted). A modified botulinum toxin with a targeting activity for a non-botulinum toxin target cell can bind to a receptor present on the non-botulinum toxin target cell, translocate into the cytoplasm, and exert its proteolytic effect on the SNARE complex of the target cell. In essence, a botulinum toxin light chain comprising an enzymatic domain is intracellularly delivered to any desired cell by selecting the appropriate targeting domain.

In some embodiments, the clostridial derivative is a botulinum toxin, which is selected from the group consisting of botulinum toxin types A, B, C₁, D, E, F, G, H, X, En and mosaic botulinum toxins. In one embodiment, the clostridial derivative of the present method is a botulinum toxin type A. The botulinum toxin can be a recombinant botulinum neurotoxin, such as botulinum toxins produced by E. coli.

The clostridial derivative, such as a botulinum toxin, for use according to the present invention can be stored in lyophilized, vacuum dried form in containers under vacuum pressure or as stable liquids. Prior to lyophilization the botulinum toxin can be combined with pharmaceutically acceptable excipients, stabilizers and/or carriers, such as, for example, albumin, or the like. Acceptable excipients or stabilizers include protein excipients, such as albumin or gelatin, or the like, or non-protein excipients, including poloxamers, saccharides, polyethylene glycol, or the like. In embodiments containing albumin, the albumin can be, for example, human serum albumin or recombinant human albumin, or the like. The lyophilized material can be reconstituted with a suitable liquid such as, for example, saline, water, or the like to create a solution or composition containing the botulinum toxin to be administered to the patient.

In some embodiments, to increase the resident time of the clostridial derivative in the joint, the clostridial derivative is provided in a controlled release system comprising a polymeric matrix encapsulating the clostridial derivative, wherein fractional amount of the clostridial derivative is released from the polymeric matrix over a prolonged period of time in a controlled manner. Controlled release neurotoxin systems have been disclosed for example in U.S. Pat. Nos. 6,585,993; 6,585,993; 6,306,423 and 6,312,708, each of which is hereby incorporated by reference in its entirety.

The therapeutically effective amount of the clostridial derivative, for example a botulinum toxin, administered according to the present method can vary according to the potency of the toxin and particular characteristics of the condition being treated, including its severity and other various patient variables including size, weight, age, and responsiveness to therapy. The potency of the toxin is expressed as a multiple of the LD₅₀ value for the mouse, one unit (U) of toxin being defined as being the equivalent amount of toxin that kills 50% of a group of 18 to 20 female Swiss-Webster mice, weighing about 20 grams each.

The therapeutically effective amount of the botulinum toxin according to the present method can vary according to the potency of a particular botulinum toxin, as commercially available Botulinum toxin formulations do not have equivalent potency units. For example, one unit of BOTOX® (onabotulinumtoxinA), a botulinum toxin type A available from Allergan, Inc., has a potency unit that is approximately equal to 3 to 5 units of DYSPORT® (abobotulinumtoxinA), also a botulinum toxin type A available from Ipsen Pharmaceuticals. In some embodiments, the amount of abobotulinumtoxinA, (such as DYSPORT®), administered in the present method is about three to four times the amount of onabotulinumtoxinA (such as BOTOX®) administered, as comparative studies have suggested that one unit of onabotulinumtoxinA has a potency that is approximately equal to three to four units of abobotulinumtoxinA. MYOBLOC®, (known as NEUROBLOC® outside the United States) a botulinum toxin type B available from Elan, currently USWorldmeds, has been reported to have a much lower potency unit relative to BOTOX®. In some embodiments, the botulinum neurotoxin can be a pure toxin, devoid of complexing proteins, such as XEOMIN® (incobotulinumtoxinA). The quantity of toxin administered, and the frequency of its administration will be at the discretion of the physician responsible for the treatment and will be commensurate with questions of safety and the effects produced by a particular toxin formulation. In some embodiments, the Clostridial derivative is selected from onabotulinumtoxinA, incobotulinumtoxinA, abotulinumtoxinA, daxibotulinumtoxinA, prabotulinumtoxinA, and rimabotulinumtoxinB.

Methods and medicaments for treating CSD related disorders and/or symptoms thereof according to the present disclosure can comprise a CGRP antagonist, a clostridial derivative, such as a botulinum toxin, or combination thereof for administration to a patient with a CSD related disorder. Administration of the CGRP antagonist, clostridial or combination thereof can be intravenous, peripheral, extracranial, oral, intramuscular, subdermal, intradermal, topical, extra-axial or extradural. The CGRP antagonist is administered in a therapeutically effective amount to alleviate, prevent or treat a CSD related disorder and/or symptoms thereof.

In some embodiments, the administration of the CGRP antagonist, the clostridial derivative such as a botulinum toxin, or combination thereof prevents the progression of a focal seizure in a generalized seizure.

In some embodiments, the combination of the CGRP antagonist and clostridial derivative allows for lower doses of both and/or each component. This results in decreased side effects. In some other embodiments, the combination therapy allows for faster relief, higher efficacy, longer therapeutic duration, or combination thereof.

### EXAMPLES

The following non-limiting examples provide those of ordinary skill in the art with possible case scenarios and specific methods to treat conditions within the scope of the present disclosure and are not intended to limit the scope of the disclosure. In the following examples administration of a CGRP antagonist in combination with a botulinum toxin can be carried out. For example, by topical application (cream or transdermal patch), subcutaneous injection, or subdermal implantation of a controlled release implant.

### Example 1

### Administration of an Exemplary Clostridial Derivative Reduces Activation of C-Fibers but not Aδ-Fibers

Female Sprague-Dawley rats weighing 210-250 g were briefly anesthetized (2% isoflurane) and injected with BoNT-A (onabotulinumtoxinA, final dose=5 units) or vehicle (normal saline). Four injections of BoNT-A (each containing 1.25 units diluted in 5 μl saline) or saline (5 μl) were made along the lambdoid (2 injection sites) and sagittal (2 injection sites) sutures.

Seven to 14 days following the injection of BoNT-A or saline, the rats were surgically prepared for single-unit recording in the trigeminal ganglion (platinum-coated tungsten microelectrodes, impedance 150-300 kΩ). A glass micropipette filled with 0.9% saline (˜1 megohm, 7 um tip) was inserted below the cortical surface for electrocorticogram recording.

A single wave of CSD was then induced by pinprick and confirmed by recording of the propagating CSD wave with the recording micropipette in the frontal cortex. Recording of ongoing discharge continued for 2-3 hours following induction of CSD.

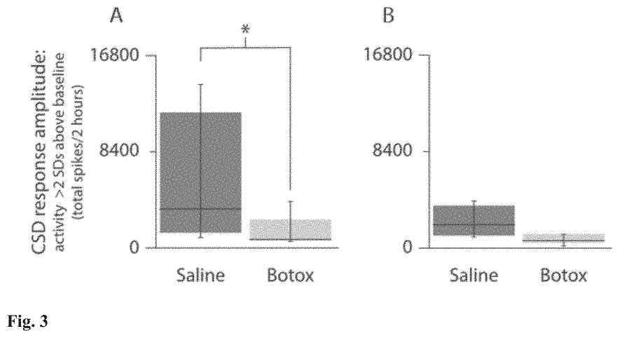

Firing rate was analyzed in 1-minute bins. A neuron was considered to have a response to CSD if its firing rate increased above baseline by at least 2 standard deviations for a period of at least 10 min (Exhibit A, FIGS. 3,4,5A). The total firing that exceeded this level was calculated as a measure of response amplitude. Response latency was also measured. Neurons from BoNT-A-treated vs saline-treated animals were compared for percentage of neurons with a CSD response (Chi-square) and response amplitude (Mann-Whitney). Aδ- and C-fiber neurons were analyzed separately.

Single unit recordings were obtained from 29 C-fibers (n=16 BoNT/A, n=13 saline) and 26 Aδ-fibers (n=15 BoNT/A, n=11 saline) in the trigeminal ganglion.

Baseline firing rates prior to CSD did not significantly differ between neurons recorded in BoNT-A vs saline-treated animals (BoNT-A, 0.7 Hz, IQR 0.3-1.2; saline, 0.8 Hz, IQR 0.3-1.7; p=0.58). There was no significant difference in the baseline firing rates between BoNT-A treated Aδ-neurons (0.7 spikes/sec [IQR: 0.5 to 1.2]) and saline treated Aδ-neurons (1.0 spikes/sec [IQR: 0.7 to 1.7]) (p=0.16). Similarly, the BoNT-A treated C-fibers (0.7 spikes/sec [IQR: 0.2 to 1.2]) were comparable to saline treated group (0.7 spikes/sec [IQR: 0.06 to 1.8]) (p=0.64).

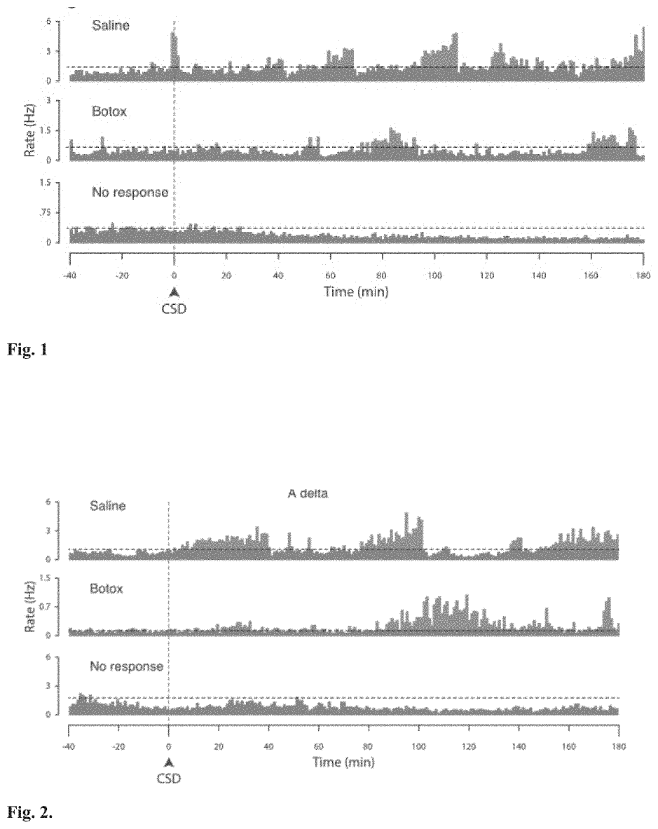

Since all neurons that were eventually classified as activated had started their increase in firing frequency by around 1 hour post-CSD, the interval of 60-180 for the analysis of response amplitude was examined. This was done to avoid comparing data from a time period in which some activated neurons are in their pre-activation stage while others are already activated. Responses are reported as the total firing (total number of spikes) during the 2-hour recording period that was more than two standard deviations above the baseline level (as analyzed in 1-minute bins). In this time period, there was a significant effect of the BoNT-A on the C-fibers, but not the Aδ-fibers (FIGS. 1-3). In the C fibers, there was a reduced level of activation in the BoNT-A treated animals, (566 spikes [IQR: 360-1147], n=6) as compared to the saline-treated animals (1990 spikes [IQR: 989 to 3723], n=4) (p=0.036). In the Aδ-neurons, no significant difference was found between the BoNT-A treated animals (636 spikes [IQR: 500 to 2403], n=5) and saline-treated animals (2742 spikes [IQR: 758 to 3975], n=3) (p=0.40). These results suggest that BoNT/A inhibited the response to CSD in unmyelinated C-fibers but not in myelinated A-delta fibers in the trigeminal ganglion.

### Example 2

### Administration of an Exemplary CGRP Antagonist Blocks Activation of Trigeminalvascular Aδ-Nociceptors and High-Threshold (HT) Neurons

It was found that treatment of rats with fremanezumab, a CGRP antagonist monoclonal antibody, blocked the activation of trigeminovascular Aδ-nociceptors and high threshold (HT) neurons but not C-fiber and wide dynamic range (WDR) neurons (data not shown).

### Example 3

### Administration of an Exemplary Clostridial Derivative in Combination with an Exemplary CGRP-Antagonist Prevents Activation and Sensitization of High Threshold (HT) and Wide Dynamic Range (WDR) Neurons

Male rats were pre-treated with 5 units of BOTOX® (onabotulinumtoxinA) or saline injected along the suture lines of the skull (5 injections; 1 unit per site) 7 to 11 days prior to electrophysiological recordings. For electrophysiological recordings, rats were anesthetized (1.2 mg/kg of urethane), paralyzed with rocuronium (1.0 ml per hour, 1 mg/ml), and treated with 0.4 mg of atropine and 1 ml of bupivocaine. All animals were ventilated throughout the experiments. Animals received Atogepant or vehicle intravenously (5 mg/kg) 1 hour before induction of cortical spreading depression. Responses to a single wave of CSD were studied using single-unit extracellular recording in 22 dura-sensitive neurons in the spinal trigeminal nucleus of the brain (11 high threshold neurons and 11 wide dynamic range neurons).

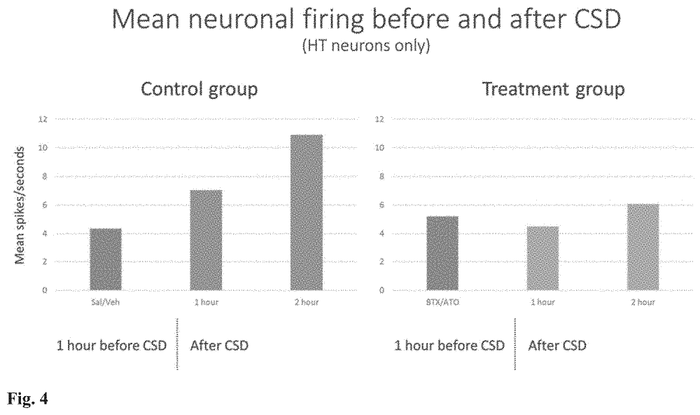

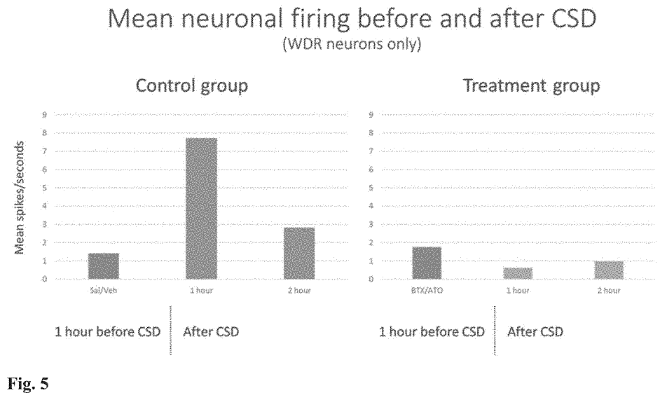

Results are summarized in Tables 1 and 2 and presented in FIGS. 4 and 5.

Table 1 shows the numbers and percentages of neurons in the spinal trigeminal nucleus that were activated by cortical spreading depression. In control animals treated with saline and vehicle, 7 of 10 neurons tested were activated in response to CSD, whereas in animals treated with Botox and Atogepant, only 1 of 12 neurons tested was activated (P=0.002). This effect was observed in both high threshold neurons and wide dynamic range neurons. These data show that Botox+Atogepant prevented the activation of spinal trigeminal neurons in response to CSD.

TABLE 1
Table 1: Numbers (%) of spinal trigeminal neurons (identified
electrophysiologically as wide dynamic range or high threshold
cells) activated by cortical spreading depression in control
animals vs. animals treated with Botox and atogepant

| | Control | | Botox + Atogepant | | Chi square |
|---|---|---|---|---|---|
| | n/N (%) | | n/N (%) | | P value |
| All neurons tested | 7/10 | (70.0%) | 1/12 | (8.3%) | 0.002 |
| High threshold | 4/5 | (80.0%) | 1/6 | (16.7%) | 0.035 |
| neurons | | | | | |
| Wide dynamic range | 3/5 | (60.0%) | 0/6 | (0%) | 0.026 |
| neurons | | | | | |

Table 2 shows that the mean firing rates of the spinal trigeminal neurons increased dramatically in response to CSD at both 1 and 2 hours post-induction in control animals but not in animals treated with Botox plus Atogepant. These data show that Botox+Atogepant pre-treatment prevents the increase in firing rate of spinal trigeminal neurons in response to cortical spreading depression in this experimental model.

TABLE 2
Table 2: Mean firing rates of trigeminovascular neurons (identified
electrophysiologically as wide dynamic range or high threshold cells)
before and after induction of cortical spreading depression (CSD)
in control animals and animals treated with Botox plus atogepant.

| | Before CSD | 1 hour after CSD | 2 hours after CSD |
|---|---|---|---|
| | (spikes/sec) | (spikes/sec) | (spikes/sec) |
| Control | 2.88 | 7.37 | 6.86 |
| (N = 10 neurons) | | (256% increase) | (238% increase) |
| Botox + Atogepant | 3.49 | 2.55 | 3.52 |
| (N = 12 neurons) | | (27% decrease) | (1% increase) |

FIG. 4 shows that the mean firing rate of high threshold spinal trigeminal neurons more than doubles in the control group 2 hours after CSD (left panel), whereas neurons in the treated group do not show this increase (right panel). FIG. 5 shows that the mean firing rate of wide dynamic range spinal trigeminal neurons increases dramatically in the control group within 1 hour of CSD (left panel), whereas neurons in the treated group do not show this increase (right panel). Thus, pre-treatment with onabotulinumtoxinA plus atogepant prevents the increased activity of high threshold and wide dynamic range spinal trigeminal neurons in response to CSD.

The results shown in Tables 1, 2 and FIGS. 4 and 5 show that the combination of onabotulinumtoxinA plus atogepant blocks the activation and firing rate of both high threshold and wide dynamic range neurons in response to CSD.

### Example 4

### Administration of an Exemplary Clostridial Derivative after Head Injury Before Onset of Symptoms Prevents TBI Associated Symptoms

Two football players are injured in a collision during a game. The first player is treated promptly after the head injury in the emergency department and neuroimaging with CT scan is normal. In the emergency room, the first player has no symptoms of headache, dizziness and difficulty concentrating. The first player's treatment comprises of 155 U onabotulinumtoxinA according to the PREEMPT paradigm, with an additional 2 injections of 5 Units into each temporalis muscle (total 20 U) and 2 injections of 5 U into each occipitalis muscle (total 20 U) for an overall total of 195 Units. Over the course of the following months, he continues to do well.

In the emergency room, the second player also has no symptoms of headache, dizziness or difficulty concentrating and neuroimaging with CT scan is normal. He does not receive any treatment in the ER and is discharged home. Over the course of the following 2 weeks, he develop very severe symptoms, including headaches, mental fogginess, and emotional outbursts, cognitive disruption, photophobia, phonophobia and sleep disorder. After 1 month of symptoms, the second player is treated with BOTOX according to the PREEMPT paradigm, with an additional 2 injections of 5 Units into each temporalis muscle (total 20 U) and 2 injections of 5 U into each occipitalis muscle (total 20 U) for an overall total of 195 Units. His headaches are lessened in severity and frequency, he experiences improved sleep and improved cognition. He continues treatment with onabotulinumtoxinA every 3 months and his symptoms continue to improve so that he does not require any additional treatment after 12 months of therapy.

### Example 5

### Administration of an Exemplary Clostridial Derivative or an Exemplary CGRP-Antagonist after Blast Injury Before Onset of Symptoms Prevents Injury Associated Symptoms

Three healthy marines are injured when a land mine explodes close to their vehicle. They sustain whiplash injuries, but no direct head trauma. Medical assessment is that all three are deemed to be at risk for post traumatic syndrome including features of concussion and post traumatic stress disorder. They are offered preventive treatment but are advised that they can wait until symptoms occur.

The first marine opts for treatment with onabotulinumtoxinA 155 units using the PREEMPT injection sites, and the second one opts for treatment with an anti-CGRP monoclonal antibody, Galcanezumab 240 mg subcutaneously administration is chosen. The third marine opts to wait for symptoms to develop. These treatments are started within the first day after the injury for both of these marines (the first and second). The third marine develops symptoms of headache, photophobia, dizziness, tinnitus and insomnia a week after the blast injury. The first two marines treated do not develop any features of a post-traumatic syndrome during the following 12 months of observation. The third marine continues with progressive symptoms of headache, photophobia, dizziness, tinnitus and insomnia for the next 4 weeks and then determines that he would like to be treated to prevent any further symptom development. He is injected with Onabot A 155 units using the PREEMPT injection sites. His headaches resolve a month after his treatment with decreased photophobia, dizziness, tinnitus and insomnia. None of these marines develops post traumatic stress disorder.

### Example 6

### Administration of an Exemplary Clostridial Derivative in Combination with an Exemplary CGRP-Antagonist after Whiplash Injury Before Onset of Symptoms Prevents Injury Associated Symptoms

A 42 year old woman has a pre-existing history of low frequency episodic migraine with visual aura. Headache symptoms were treated with ibuprofen with good results. She is driving to work and her car is struck from behind when she slows for traffic. She sustains a whiplash injury and her head hits the steering wheel as her air bag does not deploy. She develops neck pain on the day following the accident. She sees her neurologist and expresses concern that her headaches will worsen as a result of the accident. He explains that an option would be to preemptively treat her to prevent chronic migraine from developing by injecting onabotulinumtoxinA in combination with a CGRP monoclonal antibody. She agrees and undergoes onabotulinumtoxinA treatment with 155 units using the PREEMPT program and Erenumab 140 mg subcutaneous injection. No headaches occur in the following 3 months and so she does not proceed with a second treatment of either treatment modality. Four months after the accident she develops daily headaches with migraine features. As a result she goes ahead with a second treatment of onabotulinumtoxinA and Erenumab and her headaches abate. She continues this treatment for the next 12 months, with both therapeutic agents. She discontinues after this period of time as her headaches have not returned. She remains symptom free at her 18 month follow up.

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Many alterations and modifications may be made by those having ordinary skill in the art, without departing from the spirit and scope of the disclosure. Therefore, it must be understood that the described embodiments have been set forth only for the purposes of examples, and that the embodiments should not be taken as limiting the scope of the following claims. The following claims are, therefore, to be read to include not only the combination of elements which are literally set forth, but all equivalent elements for performing substantially the same function in substantially the same way to obtain substantially the same result. The claims are thus to be understood to include those that have been described above, those that are conceptually equivalent, and those that incorporate the ideas of the disclosure.

1. A method for treating, alleviating, preventing, or reducing the intensity or frequency of occurrence of a disease or condition associated with cortical spreading depression (CSD) and/or symptoms thereof in a patient in need thereof, comprising administering to the patient an antagonist of calcitonin gene-related peptide (CGRP-antagonist), a clostridial derivative, or combination thereof.

2. A method for treating, alleviating, preventing, or reducing the intensity or frequency of occurrence of a disease or condition associated with cortical spreading depression (CSD) and/or symptoms thereof in a patient in need thereof, comprising administering to the patient;
(a) an antagonist of calcitonin gene-related peptide (CGRP-antagonist); and optionally,
(b) a clostridial derivative.

3. (canceled)

4. A method for treating, alleviating, preventing, or reducing the intensity or frequency of occurrence of a disease or condition associated with cortical spreading depression (CSD) and/or symptoms thereof in a patient in need thereof, comprising administering to the patient a clostridial derivative.

5. The method of claim 1, the method comprising administering to the patient a clostridial derivative and a CGRP-antagonist.

6. The method of claim 1, wherein said disease or condition is selected from stroke, subarachnoid and intracranial hemorrhage, traumatic brain injury, seizures, spreading depolarizations related to head injury/head trauma (concussion), post-traumatic stress disorder, fibromyalgia, and tinnitus (either associated with or without another disorder).

7. The method of claim 1, wherein the CSD-related symptoms are other than headache, migraine or migraine variants.

8. (canceled)

9. The method according claim 1, wherein said clostridial derivative is a botulinum toxin of serotype A, B, C, D, E, F, G, H, X, or En.

10. The method according to claim 9, wherein the clostridial derivative is onabotulinumtoxinA.

11. The method according to claim 1, wherein said CGRP-antagonist is an anti-calcitonin gene-related peptide receptor antibody selected from the group consisting of galcanezumab, fremanezumab, eptinezumab, and erenumab.

12. (canceled)

13. The method according to claim 1, wherein said antagonist of CGRP receptor is selected from ubrogepant, atogepant, rimegepant or a pharmaceutically acceptable salt thereof.

14-24. (canceled)

25. The method according to claim 11, wherein said anti-CGRP antibody is erenumab, and wherein erenumab is administered subcutaneously at a dose of about 5 mg to about 500 mg every one, two, three, four, five, six, seven, eight, nine or ten weeks.

26-38. (canceled)

39. The method according to claim 11, wherein said anti-CGRP antibody is galcanezumab, and wherein galcanezumab is administered subcutaneously at a dose of about 10 mg to about 500 mg every one, two, three, four, five, six, seven, eight, nine or ten weeks.

40-52. (canceled)

53. The method according to claim 11, wherein said anti-CGRP antibody is fremanezumab, and wherein fremanezumab is administered subcutaneously at a dose of about 100 mg to about 1000 mg every one, two, three, four, five, six, seven, eight, nine or ten weeks.

54-69. (canceled)

70. The method according to claim 11 wherein said anti-CGRP antibody is eptinezumab, and wherein eptinezumab is administered subcutaneously at a dose of about 50 mg to about 1000 mg every one, two, three, four, five, six, seven, eight, nine or ten weeks.

71-84. (canceled)

85. The method according to claim 10, wherein said onabotulinumtoxinA is administered at a dose of:
about 1 to about 1,000 units every one, two, three, four, five, six, seven, eight, nine, ten, eleven, twelve, thirteen, fourteen, fifteen or sixteen weeks; or
about 1 unit, about 2 units, about 3 units, about 4 units, about 5 units, about 6 units, about 7 units, about 8 units, about 9 units or about 10 units every one, two, three, four, five, six, seven, eight, nine, ten, eleven, twelve, thirteen, fourteen, fifteen or sixteen weeks; or
about 10 units, about 15 units, about 20 units, about 25 units, about 30 units, about 40 units, about 45 units, about 50 units, about 55 units or about 60 units every one, two, three, four, five, six, seven, eight, nine, ten, eleven, twelve, thirteen, fourteen, fifteen or sixteen weeks; or
about 25 units, about 50 units, about 75 units, about 100 units, about 125 units, about 150 units, about 175 units, about 200 units, about 225 units or about 250 units every one, two, three, four, five, six, seven, eight, nine, ten, eleven, twelve, thirteen, fourteen, fifteen or sixteen weeks.

86-96. (canceled)

97. The method according to claim 1 wherein said antagonist of CGRP receptor is ubrogepant or a pharmaceutically acceptable salt thereof, and ubrogepant is administered at a dose of about 5 to about 500 mg per day.

98. (canceled)

99. The method according to claim 1 wherein said antagonist of CGRP receptor is atogepant or a pharmaceutically acceptable salt thereof, and atogepant is administered at a dose of about 5 to about 500 mg per day.

100-102. (canceled)

103. The method according to claim 4, wherein the method alleviates, reduces the intensity or reduces the occurrence or prevents traumatic brain injury associated symptoms in the patient, and wherein the administering is carried out within six months after the traumatic brain injury.

104. (canceled)

105. (canceled)

106. The method of claim 103, wherein the administering is carried out prior to onset of at least one of the traumatic brain injury associated symptom.

107. The method of claim 103, wherein the administering is carried out within one month after the onset of a first traumatic brain injury associated symptom.

108. (canceled)
